(19) United States
(12) Patent Application Publication
(10) Pub. No.: US 2004/0247674 A1
(43) Pub. Date: Dec. 9, 2004

(54) Drug delivery system

(76) Inventors: Timo Haapakumpu, Littoinen (FI); Juha Ala-Sorvari, Turku (FI); Marko Aaltonen, Aura (FI); Antti Keinanen, Turku (FI); Manja Ahola, Piikkio (FI)

Correspondence Address: JAMES C. LYDON, 100 DAINGERFIELD ROAD, SUITE 100, ALEXANDRIA, VA, 22314, US

(21) Appl. No.: 10/487,992

(22) PCT Filed: Aug. 27, 2002
(86) PCT No.: PCT/FI02/00692

Related U.S. Application Data
(60) Provisional application No. 60/315,972, filed on Aug. 31, 2001.

(51) Int. Cl. A61K 9/24

(52) U.S. Cl. 424/471

(57) ABSTRACT

A delivery system including at least one core and a membrane. The core and the membrane include an elastomer composition containing, e.g., poly(dimethylsiloxane), a siloxane-based elastomer having 3,3,3-trifluoropropyl groups attached to the Si-atoms of the siloxane units and/or poly(alkylene oxide) groups, present as alkoxy-terminated grafts or blocks linked to the polysiloxane units by silicon-carbon bonds, or as a mixture of these forms. The delivery system is preferably an implant or an interuterine, intracervical or intravaginal system.

### FIELD OF THE INVENTION

This invention relates to a delivery system comprising a core and a membrane encasing said core wherein said core and membrane consist essentially of a same or different elastomer composition.

### BACKGROUND OF THE INVENTION

The patents U.S. Pat. No. 6,056,976 and U.S. Pat. No. 6,299,027 and the pending application serial number U.S. Ser. No. 09/701,547, filed Nov. 30, 2000 (equivalent: WO 00/00550) are incorporated by reference.

Polysiloxanes, such as poly(dimethylsiloxane) (PDMS), are highly suitable for use as a membrane or a matrix regulating the permeation of active agents in various active agent forms, in particular in implants and intra-uterine systems (IUS). Polysiloxanes are physiologically inert, and a wide group of active agents are capable of penetrating polysiloxane membranes, which also have the required mechanical properties.

Applicant's pending application Ser. No. 09/701,547, filed Nov. 30, 2000 discloses an elastomer composition comprising poly(alkylene oxide) groups and poly(alkylene oxide) groups being present in the elastomer or polymer as alkoxy-terminated grafts of polysiloxane units, or as blocks, the said blocks or grafts being linked to the polysiloxane units by silicon-carbon bonds, or as a mixture of these forms. This application also discloses the method of preparation of such elastomers.

Applicant's granted patent U.S. Pat. No. 6,056,976 discloses an elastomer that is a siloxane-based elastomer comprising 3,3,3-trifluoropropyl groups attached to the Si-atoms of the siloxane units, and the release rate of said therapeutically active agent of said delivery system is regulated by the amount of said 3,3,3-trifluoropropyl groups.

Several publications disclose delivery systems that are capable of releasing more than one therapeutically active agent. For example, the patent U.S. Pat. No. 5,972,372 discloses a vaginal ring that comprises a body comprising a first polymeric material and a hollow internal channel. The ring further comprises a drug-containing core that is placed in said internal channel and is made of a second polymeric material. Said polymeric material may for example be silicone elastomers such as PDMS or its derivative containing fluoro-groups. This document does however not disclose a core-membrane structure.

The patent U.S. Pat. No. 5,443,461 discloses a diffusional delivery system that is constructed in two or more compartments, each containing a therapeutically active agent. These active agents are released independently from each other. The wall segments between the compartments may be manufactured from thermoplastic elastomers, for example. The active agent is formulated in a composition that includes a dilution agent such as polymer blends. Polyethylene glycol blends are given as an example of suitable blends.

The patent U.S. Pat. No. 5,496,557 presents a delivery system for controlled delivery of an active substance comprising a hollow space enclosed by a wall and filled with said active substance. The wall is made of a biodegradable polymer and only one example of the filling is given, namely dispersion of an active substance in castor oil. This system does thus not disclose a core made of an elastomer. The system is further coated with non-permeable biodegradable polymer and the rate of diffusion of the active substance is controlled by the surface of the wall not covered by said non-permeable polymer. A problem that could occur with this kind of system is that if the wall is broken, the active substance is released in a non-controlled manner. Such release could lead to serious problems due to the side effects of the active substances or an intoxication by the active substances.

### OBJECTS AND SUMMARY OF THE INVENTION

An object of this invention is to provide a delivery system capable of releasing at least two different active agents at the same time and at constant, pre-defined rates.

A further object of the invention is to provide a delivery system that, even if damaged, would not cause any danger to the subject.

Furthermore, the invention aims to provide a delivery system that is easy and cost-effective to produce.

### DETAILED DESCRIPTION OF THE INVENTION

The invention is disclosed in the appended claims.

The system according to the invention comprising a core and a membrane encasing said core, wherein said core and membrane consist essentially of a same or different elastomer composition, is characterized in that the core comprises at least two therapeutically active agents each having a release rate.

The core and the membrane are thus essentially made of a same or different elastomer composition that is described further below. In this application, the term “elastomer composition” may stand for one single elastomer, or the elastomer composition may be made up of two elastomers that are interlaced, one inside the other.

The elastomer composition used in the membrane is such that it allows the pre-determined, constant release rates of the active agents. The first object of the invention is thus obtained by the choice of the elastomer composition. Secondly, the core consists essentially of an elastomer composition, that is, the core is an elastomer matrix wherein the active agents are dispersed. Therefore, even if the membrane encasing the core would be damaged, the active agents would not be released in a completely uncontrolled manner causing above-mentioned problems to the subject. The elastomer composition of the core is thus chosen such that the release rates of the active agents from the core are higher than the release rates through the membrane but low enough to avoid any problems. The release rates can thus be controlled by the membrane alone or by the membrane together with the core. It is also possible that the release rate is mainly controlled by the core and that the membrane performs only the final control of the release rate.

The delivery system according to the invention may be an implant, an intrauterine system, an intracervical system or an intravaginal system. The manufacturing of such systems is discussed below, even though it is well known in the art. The shape and size of the system may also be freely chosen by the person skilled in the art. It is also evident that the systems according to the invention may be applied to humans as well as to animals. When the delivery system is for example an intrauterine system, it may further comprise a body forming the structure of the system. In this case, the core-membrane-structure of the system is hollow so that it can be positioned over the body of the system. The body may have the form of T, S or 7.

According to an embodiment of the invention, the core consists of one part comprising said at least two therapeutically active agents. According to another embodiment of the invention, the core consists of at least two parts each part comprising at least one of said at least two therapeutically active agents. The elastomer compositions of said parts are chosen according to the release rates desired and can be the same or different in each part. According to the embodiment in which the core consists of two or more parts, the parts may be either positioned next to each other or in such a way that one part of a core encases at least partly another part of the core. Any combination of structure is naturally possible and within the scope of the invention. An advantage of the use of several parts is that the release rates are more easily controllable since there is no interaction between the active agents.

According to a further embodiment of the invention, the membrane consists of at least two layers, each layer having a certain thickness. The thickness of the layers may be the same or different and the elastomer compositions used in each layer may also be the same or different. The membranes encasing each above-mentioned part of the core may also be identical or different in either the elastomer composition or the structure of the membrane (one or several layers). The combination of different layer of membrane either in thickness or in material or both, gives a further possibility for controlling the release rates of the active agents.

According to an embodiment, the system according to the invention further comprises a space separating at least two of the said at least two parts of the core and/or at least one separation membrane separating at least two of the said at least two parts of the core, said separation membrane consisting essentially of an elastomer composition. It is for example possible to produce a system according to the invention having a core consisting of three parts A, B and C, the parts A and B being separated by a space and the parts B and C being separated by a membrane. A system wherein the parts A and B are next to each other without a space or a membrane between them and the parts B and C are separated by a membrane, or a system wherein the parts A and B are separated by a membrane consisting of a first elastomer composition and the parts B and C are separated by a membrane consisting of a second elastomer composition different from the first elastomer composition, is also within the scope of this invention, as well as any other combination.

According to a further embodiment of the invention, the separation membranes are permeable or impermeable to at least one of the therapeutically active agents. It is of course possible to use a membrane that is permeable to a first active agent but impermeable to a second active agent.

According to a preferred embodiment of the invention, the elastomer compositions mentioned above, namely the elastomer compositions of the core, the membrane and the separation membrane, are the same or different and selected from the group consisting of

an elastomer composition comprising poly(dimethylsiloxane),

an elastomer composition comprising a siloxane-based elastomer comprising 3,3,3-trifluoropropyl groups attached to the Si-atoms of the siloxane units,

an elastomer composition comprising poly(alkylene oxide) groups, said poly(alkylene oxide) groups being present as alkoxy-terminated grafts or blocks linked to the polysiloxane units by silicon-carbon bonds, or as a mixture of these forms, and

a combination of at least two thereof.

According to an embodiment of the invention, in the siloxane-based elastomer 1 to approximately 50% of the substituents attached to the Si-atoms of the siloxane units are 3,3,3-trifluoropropyl groups.

According to another embodiment of the invention, the poly(alkylene oxide) groups mentioned above are poly(ethylene oxide) groups.

The above-mentioned elastomer compositions are discussed below in further detail.

According to yet another embodiment of the invention, the release rates of the at least two therapeutically active agents are identical or different. According to a preferred embodiment of the invention, the therapeutically active agent is a hormone, such as a progestin, an estrogen, an antiprogestin or an androgen. The system may also include any other therapeutically active substance that is suitably associated with a given hormone or other active agent. Some examples of suitable therapeutically active agents are given below.

According to an embodiment of the invention, the release rates of the therapeutically active agents in an intrauterine, intracervical or intravaginal system are 0,1-300 μg/day. According to another embodiment of the invention, the release rates of the active agents in an implant are 0,1-300 μg/day, these examples being given for hormones.

Any combination of the embodiments mentioned above is possible and within the scope of this invention, and a person skilled in the art will be able to find the most suitable combination for a particular use.

The preparation of the system according to the invention is obvious to a person skilled in the art. Indeed, the system may be manufactured by extrusion or molding, for example. The preparation is further discussed below.

The Elastomer Compositions

One of the elastomers suitable for use in the system according to this invention, particularly for use in the membrane of the system, is a siloxane-based elastomer comprising 3,3,3-trifluoropropyl groups attached to the Si-atoms of the siloxane units.

The term “siloxane-based elastomer” shall be understood to cover elastomers made of poly(disubstituted siloxanes) where the substituents mainly are lower alkyl, preferably alkyl groups of 1 to 6 carbon atoms, or phenyl groups, wherein said alkyl or phenyl can be substituted or unsubstituted. A widely used and preferred polymer of this kind is poly(dimethylsiloxane) (PDMS).

According to the invention, a certain amount of the substituents attached to the Si-atoms of the siloxane units in the elastomer shall be 3,3,3-trifluoropropyl groups. Such an elastomer can be achieved in different ways. According to one embodiment, the elastomer can be based on one single crosslinked siloxane-based polymer, such as a poly(dialkyl siloxane) where a certain amount of the alkyl groups at the Si-atoms are replaced by 3,3,3-trifluoropropyl groups. A preferred example of such polymers is poly(3,3,3-trifluoropropyl methyl siloxane) the structure of which is shown as Compound I below.

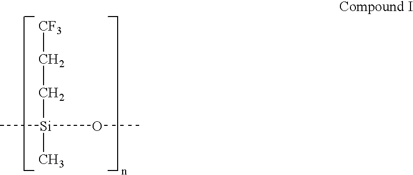

A polymer of this kind, in which approximately 50% of the methyl substituents at the Si-atoms are replaced by 3,3,3-trifluoropropyl groups, is commercially available. The term “approximately 50%” means that the degree of 3,3,3-trifluoropropyl substitution is in fact somewhat below 50%, because the polymer must contain a certain amount (about 0.15% of the substituents) of cross-linkable groups such as vinyl or vinyl-terminated groups. Similar polymers having lower substitution degree of 3,3,3-trifluoropropyl groups can easily be synthesized.

The retarding effect of the 3,3,3-trifluoropropyl groups on the permeation of active agents across a membrane of the elastomer is dependent on the amount of these groups. Furthermore, the effect is highly dependent on the active agent used. If the elastomer is made of one single polymer only, it is necessary to prepare and use polymers with different amounts of 3,3,3-trifluoropropyl groups for different active agents.

According to another embodiment, which is particularly preferred if suitable elastomers for several different active agents are needed, is to crosslink a mixture comprising a) a non-fluorosubstituted siloxane-based polymer and b) a fluorosubstituted siloxane-based polymer, where said polymer comprises 3,3,3-trifluoropropyl groups attached to the Si-atoms of the siloxane units. The first ingredient of the mixture, the non-fluorosubstituted polymer, can be any poly(disubstituted siloxane) where the substituents mainly are lower alkyl, preferably alkyl groups of 1 to 6 carbon atoms, or phenyl groups, wherein said alkyl or phenyl can be substituted or unsubstituted. The substituents are most preferably alkyl groups of 1 to 6 carbon atoms. A preferred non-fluorosubstituted polymer is PDMS. The second ingredient of the mixture, the fluoro-substituted polymer, can for example be a poly(dialkyl siloxane) where a certain amount of the alkyl groups at the Si-atoms are replaced by 3,3,3-trifluoropropyl groups. A preferred example of such polymers is poly(3,3,3-trifluoropropyl methyl siloxane) as mentioned above. A particularly preferable polymer of this kind is a polymer having as high amount of 3,3,3-trifluoropropyl substituents as possible, such as the commercially available polymer, in which approximately 50% of the methyl substituents at the Si-atoms are replaced by 3,3,3-trifluoropropyl groups. An elastomer with great permeation retarding effect can be achieved by using exclusively or mainly the aforementioned polymer. Elastomers with less retarding influence on the permeation of the active agent can be obtained by using mixtures with increasing amounts of the non-fluorosubstituted siloxane-based polymer.

Another elastomer that can be used in this invention comprises poly(alkylene oxide) groups so that the poly(alkylene oxide) groups are present in the said elastomer either as alkoxy-terminated grafts of polysiloxane units or as blocks, the said grafts or blocks being linked to the polysiloxane units by silicon-carbon bonds. The poly(alkylene oxides) may also be present as a blend of the options mentioned. The second elastomer may be a siloxane-based elastomer, suitably a poly(dimethyl siloxane)-based elastomer. The said second elastomer may possibly also comprise poly(alkylene oxide) groups. These poly(alkylene oxide) groups may also be present either as alkoxy-terminated grafts of poly(dimethyl siloxane) units or as blocks, the said grafts or blocks being linked to the poly(dimethyl siloxane) units by silicon-carbon bonds. The poly(alkylene oxides) may also in this elastomer be present as a blend of the options mentioned above.

According to an embodiment of the invention, the elastomer composition may be a blend which comprises a siloxane-based elastomer, which is, for example, made up of PDMS, and at least one straight-chain polysiloxane copolymer which comprises poly(alkylene oxide) groups. In this case the poly(alkylene oxide) groups are present in the said polymer either as alkoxy-terminated grafts of polysiloxane units or as blocks, the said grafts or blocks being linked to the polysiloxane units by silicon-carbon bonds. The poly(alkylene oxide) groups may, of course, also be present in the polymer as a blend of the forms mentioned. In this embodiment, also the siloxane-based elastomer may comprise poly(alkylene oxide) groups, in which case these poly(alkylene oxide) groups are present in the elastomer either as alkoxy-terminated grafts of polysiloxane units or as blocks, the said blocks or grafts being linked to the polysiloxane units by silicon-carbon bonds. The poly(alkylene oxide) groups may also be present as a blend of the forms mentioned.

Of course, the elastomer composition may also be made up of two elastomers interlaced one inside the other, as above, and at least one straight-chain polysiloxane copolymer which comprises poly(alkylene oxide) groups.

The poly(alkylene oxide) groups of the elastomer composition may suitably be, for example, poly(ethylene oxide) groups (PEO groups).

The polysiloxane units of the elastomer composition are preferably groups having the formula

—(SiR′R″O)_{q}SiR′R″

where R′ and R″ are

partly free groups, which are the same or different and which are a lower alkyl group, or a phenyl group, in which case the said alkyl or phenyl groups may be substituted or unsubstituted, or alkoxy-terminated poly(alkylene oxide) groups having the formula

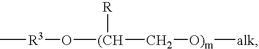

where alk is a lower alkyl group, suitably methyl, R is hydrogen or a lower alkyl, m is 1 . . . 30, and R³ is a straight or branched C₂-C₆ alkyl group,

partly bonds, formed from the hydrogen or alkylene groups, to other polymer chains in the elastomer, and

possibly partly unreacted groups, such as hydrogen, vinyl or vinyl-terminated alkene, and

q is 1 . . . 3000.

The term “lower alkyl” stands here and generally in the description of the present invention for C₁-C₆ alkyl groups.

The above-mentioned free R′ and R″ groups are suitably a lower alkyl group, preferably methyl.

The term “poly(alkylene oxide) group” means that said group comprises at least two alkyl ether groups successively connected to each other.

According to a preferred embodiment, the poly(alkylene oxide) groups are present in the elastomer in the form of poly(alkylene oxide) blocks having the formula

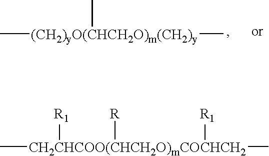

where R is hydrogen, a lower alkyl or a phenyl,

R₁ is hydrogen or a lower alkyl, y is 2 . . . 6, and m is 1 . . . 30.

Preferable combinations of elastomers are PDMS with poly(ethylene oxide)-PDMS and PDMS with fluorosubstituted PDMS.

The elastomer composition preferably comprises a filler, such as amorphous silica, in order to give a sufficient strength for the membrane made from said elastomer. It is also possible to include other additives, while taking into account that they need to be biocompatible and harmless to the subject.

The methods for the preparation of these elastomers are given in the applicant's above-mentioned patents and patent applications.

Further examples of suitable materials include polyethylene, polypropylene, polymethylpentene ethylene/propylene copolymers, ethylene/ethyl acrylate copolymers, ethylene/vinyl acetate copolymers, polycarbonate, polytetrafluoroethylene (PTFE), fluoroethylenepropylene (FEP), polyvinylidene fluoride (PVDF), polyvinylacetate, polystyrene, polyamides, polyurethane, polybutadiene, polyisoprene, chlorinated polyethylene, polyvinyl chloride, vinyl chloride copolymers with vinyl acetate, poly(methacrylate), polymethyl (meth)acrylate, poly(vinylidene) chloride, poly(vinylidene) ethylene, poly(vinylidene) propylene, polyethylene terephthalate, ethylene vinylacetate, a polyhydroxy alkoanate poly(lactic acid), poly(glycolic acid), poly(alkyl 2-cyanoacrylates), polyanhydrides, polyorthoesters, ethylene/vinyl alcohol copolymer, ethylene/vinyl acetate/vinyl alcohol terpolymer; ethylene/vinyloxyethanol copolymer, ethylene/vinyl/acetate copolymers, ethylene vinyl/alcohol copolymers, hydrophilic polymers such as the hydrophilic hydrogels of esters of acrylic and methacrylic acids, modified collagen, cross-linked polyvinyl alcohol, cross-linked, partially hydrolyzed polyvinyl acetate, silicone elastomers, especially the medical grade polydimethyl siloxanes, polyvinylmethylsiloxanes, other organopolysiloxanes, polysiloxane, neoprene rubber, butyl rubber, epichlorohydrin rubbers, hydroxyl-terminated organopolysiloxanes of the room temperature vulcanizing type which harden to elastomers at room temperature following the addition of cross-linking agents in the presence of curing catalysts, two-component dimethylpolysiloxane compositions which are platinum catalysed at room temperature or under elevated temperatures and capable of addition cross-linking as well as mixtures thereof.

Manufacture of the Implants

The implants according to this invention can be manufactured in accordance with standard techniques. The therapeutically active agent is mixed with the core matrix elastomer composition, processed to the desired shape by moulding, casting, extrusion, or other appropriate methods. The membrane layer(s) can be applied onto the core according to known methods such as by mechanical stretching, swelling or dipping. Reference is made to the US-patents U.S. Pat. No. 3,832,252, U.S. Pat. No. 3,854,480 and U.S. Pat. No. 4,957,119. An especially suitable method for preparation of the implants is disclosed in the Finnish patent FI 97947. This patent discloses an extrusion technology where prefabricated rods containing the active ingredient are coated by an outer membrane. Each such rod is, for example, followed by another rod without any active ingredient. The formed string is cut at the rods that contain no active agent. In this way, no special sealing of the ends of the implant is necessary.

Manufacture of the Intrauterine, Intravaginal and Intracervical Systems

The intra-uterine system can be made according to well-known technology. A preferable intrauterine system (IUS, intrauterine system), intravaginal system or intracervical system in common use is a T-shaped body made of plastic material such as polyethene. The body consists of an elongate member (stem) having at one end a transverse member comprising two wings. The elongate member and the transverse member form a substantially T-shaped piece when the system is positioned in the uterus. The system has an attached thread long enough to protrude out of the cervical canal when the system is in position in the uterus. The system may also have any other shape, such as 7, S, omega, ring or C. IUS:s releasing active agents have a active agent reservoir (corresponding to the present core or core-membrane) adjusted around the elongate member. It is also possible to adjust one reservoir in one part of the IUS and another reservoir to another part of the IUS. This active agent reservoir is the delivery system according to this invention, that is, a core encased in a membrane. The intrauterine, intravaginal and intracervical systems according to the invention may thus also comprise a body wherein the system comprising said core and membrane is attached.

A T-shaped intrauterine system is traditionally manufactured by first forming the body and the reservoir separately, then positioning the reservoir on the body for example by pulling and lastly by forming a membrane over the reservoir, thus forming a core-membrane-structure.

Therapeutically Active Agents

Representative examples of therapeutically active agents that may be suitable for the present invention include (grouped by therapeutic class):

Antihypertensives such as hydralazine, minoxidil, captopril, enalapril, clonidine, prazosin, debrisoquine, diazoxide, guanethidine, methyldopa, reserpine, trimethaphan, nifedipine and isradipine;

Calcium channel blockers such as diltiazem, felodipine, amlodipine, nitrendipine, nifedipine and verapamil;

Antiarrhyrthmics such as amiodarone, flecainide, disopyramide, procainamide, mexiletene quinidine, lorcainide and bepridil;

Antiangina agents such as glyceryl trinitrate, erythrityl tetranitrate, pentaerythritol tetranitrate, mannitol hexanitrate, perhexilene, isosorbide dinitrate nicorandil and nicardipine;

β-adrenergic blocking agents such as alprenolol, atenolol, bupranolol, carteolol, labetalol, metoprolol, nadolol, nadoxolol, oxprenolol, pindolol, propranolol, sotalol, timolol tirnolol maleate, bisoprolol, celiprolol and betaxolol;

Cardiotonic glycosides such as digoxin and other cardiac glycosides and theophylline derivatives;

Adrenergic stimulants such as adrenaline, ephedrine, fenoterol, isoprenaline, orciprenaline, rimeterol, salbutamol, salmeterol, terbutaline, dobutamine, phenylephrine, phenylpropanolamine, pseudoephedrine and dopamine;

Vasodilators such as cyclandelate, isoxsuprine, papaverine, dipyridamole, isosorbide dinitrate, phentolamine, nicotinyl alcohol, co-dergocrine, nicotinic acid, glyceryl trinitrate, pentaerythritol tetranitrate xanthinol, vincamine and nimodipine;

Antimigraine preparations such as ergotamine, dihydroergotamine, methysergide, pizotifen sumatriptan and flumendroxon;

Anticoagulants and thrombolytic agents such as warfarin, ticlopidine, iloprost, dicoumarol, low molecular weight heparins such as enoxaparin, streptokinase and its active derivatives;

Hemostatic agents such as aprotinin, tranexamic acid and protamine;

Analgesics and antipyretics including the opiold analgesics such as buprenorphine, dextromoramide, dextropropoxyphene, fentanyl, alfentanil, sufentanil, hydromorphone, methadone, morphine, oxycodone, papaveretum, pentazocine, pethidine, phenoperidine, codeine, dihydrocodeine, sufentanil and tilidine and non-narcotic analgesics such as flufenamic acid, indomethacin, ibuprofen, ketoprofen, tramadol, diflunisal, rimazolium, acetylsalicylic acid (aspirin), paracetamol, and phenazone;

Neurotoxins such as capsaicin;

Neuroleptics such as butyrophenone derivatives, e.g. haloperidol, or bacteriostatics and/or fungistats, such as nystatin or metronidazole;

Hypnotics and sedatives such as the barbiturates amylobarbitone, butobarbitone and pentobarbitone and other hypnotics and sedatives such as chloral hydrate, chlormethiazole, hydroxyzine and meprobamate;

Antianxiety agents such as the benzodiazepines alprazolam, bromazepam, chlordiazepoxide, clobazam, chlorazepate, diazepam, flunitrazepam, flurazepam, lorazepam, nitrazepam, oxazepam, temazepam triazolam and buspirone;

Neuroleptic and antipsychotic drugs such as the phenothiazines, chlorpromazine, fluphenazine, pericyazine, perphenazine, promazine, thiopropazate, thioridazine, trifluoperazine; and butyrophenone, droperidol and haloperidol; and other antipsychotic drugs such as pimozide, thiothixene;

Antidepressants including the bicyclic derivatives such as nomifensine, sertraline and trazodone, tricyclic antidepressants such as amitryptyline, clomipramine, desipramine, dothiepin, doxepin, imipramine, nortriptyline, opipramol, protriptyline and trimipramine and the tetracyclic antidepressants such as mianserin and the monoamine oxidase inhibitors such as isocarboxazid, phenelizine, tranylcypromine and moclobemide and selective serotonin re-uptake inhibitors such as fluoxetine, paroxetine, citalopram, fluvoxamine and sertraline;

CNS stimulants such as caffeine, methylphenidate, nizophenone and 3-(2-aminobutyl) indole;

Anti-Alzheimer's agents such as tacrine, physostigmine and olanzapine;

Anti-Parkinson's agents such as amantadine, benserazide, carbidopa, levodopa, benztropine, biperiden, benzhexol, procyclidine, seleginil, entacapone and dopamine-2 agonists such as S(−)-2-(N-propyl-N-2-thienylethylamino)-5-hydroxytetralin;

Anticonvulsants such as phenyloin, valproic acid, primidone, phenobarbitone, methylphenobarbitone and carbamazepine, ethosuximide, methsuximide, phensuximide, sulthiame and clonazepam;

Antiemetics and antinauseants such as the phenothiazines prochloperazine, thiethylperazine and 51TF-3 receptor antagonists such as ondansetron and granisetron, as well as dimenhydrinate, diphenhydramine, metoclopramide, domperidone, hyoscine, hyoscine hydrobromide, hyoscine hydrochloride, clebopride and brompride;

Anti-inflammatory agents including their racemic mixtures or individual enantiomers where applicable, preferably which can be formulated in combination with dermal penetration enhancers, such as ibuprofen, flurbiprofen, ketoprofen, aceclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol, ketoralac, flufenisal, salsalate, triethanolamine salicylate, aminopyrine, antipyrine, oxyphenbutazone, apazone, cintazone, flufenamic acid, clonixeril, clonixin, meclofenamic acid, flunixin, coichicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, carprofen, mesalazine and triflumidate;

Antirheumatoid agents such as penicillamine, aurothioglucose, sodium aurothiomalate, methotrexate and auranofin;

Muscle relaxants such as baclofen, diazepam, cyclobenzaprine hydrochloride, dantrolene, methocarbamol, orphenadrine and quinine;

Agents used in gout and hyperuricaernia such as allopurinol, colchicine, probenecid and sulphinpyrazone;

Hormones such as 3-methoxy-17α-ethynyl-1,3,5(10)-estratrien-17-ol (mestranol), 3-hydroxy-1,3,5(10)-estratrien-17-one (estrone), 17β-estradiol, estriol, ethynylestradiol, 4-pregnene-3,20-dione (progesterone), d-13-ethyl-17α-ethynyl-17β-hydroxy-4-gonen-3-one (d-norgestrel) and the esters thereof, 17α-ethynyl-19-nortestosterone (norethisterone) and the esters thereof, 6-chloro-17-hydroxy-1α, 2α-methylenepregna-4,6-diene-3,20-dione (cyproterone) and the esters thereof, 19-norhydroxyprogesterone and the esters thereof, 6-chloro-17-acetoxy-pregna-4,6-diene-3,20-dione (chlormadinone acetate), 15,16α-methylene- and 15,16α-methylene-17β-hydroxy-18-methyl-17α-ethynyl-4-estre n-3-one, 17α-acetoxy-6α-methylprogesterone (medroxy-progesterone acetate), 9β,10α-pregna-4,6-diene-3,20-dione (dydrogesterone), estradiol-3-methyl ether diethylstilbestrol, 17α-ethynylestrene-3β,17β-diol diacetate, 17α-ethynel-11β-methyl-4 estrene 3β,17β-diol 3,17-diacetate, 17α-acetoxy-11β-methyl-19-norpregn-4-en-3-one, testosterone, testosterone propionate, testosterone phenylacetate and related androgens, allyloestrenol, lynoestrenol, norgestrel, norethyndrel, norethisterone, norethisterone acetate, gestodene, levonorgestrel, medroxyprogesterone, megestrol, testosterone, methyltestosterone, clostebol acetate, drostanolone, furazabol, nandrolone oxandrolone, stanozolol, trenbolone acetate, dihydro-testosterone, 17-α-methyl-19-nortestosterone, norethindrone, etonogestrel, desogestrel and fluoxymesterone;

Adrenal cortical hormones such as desoxycorticosterone acetate, prednisolone;

Antiandrogens such as cyproterone acetate, flutamide, nilutamide and danazol;

Antiestrogens such as tamoxifen, toremifene, clomifene and epitiostanol;

Aromatase inhibitors such as letrozole, exemestane and 4-hydroxy-androstenedione and its derivatives;

5-α reductase inhibitors such as finasteride, turosteride;

Corticosteroids such as betamethasone, betamethasone valerate, cortisone, dexamethasone, dexamethasone 21-phosphate, fludrocortisone, flumethasone, fluocinonide, fluocinonide desonide, fluocinolone, fluocinolone acetonide, fluocortolone, halcinonide, halopredone, hydrocortisone, hydrocortisone 17-valerate, hydrocortisone 17-butyrate, hydrocortisone 21-acetate, methylprednisolone, prednisolone, prednisolone 21-phosphate, prednisone, triamcinolone, triamcinolone acetonide;

Steroidal anti inflammatory agents such as cortodoxone, fludroracetonide, fludrocortisone, difluorsone diacetate, flurandrenolone acetonide, medrysone, amcinafel, amcinafide, betamethasone and its other esters, chloroprednisone, clorcortelone, descinolone, desonide, dichlorisone, difluprednate, flucloronide, flumethasone, flunisolide, flucortolone, fluoromethalone, fluperolone, fluprednisolone, meprednisone, methylmeprednisolone, paramethasone, cortisone acetate, hydrocortisone cyclopentylpropionate, cortodoxone, flucetonide, fludrocortisone acetate, flurandrenolone acetonide, medrysone, aincinafal, amcinafide, betamethasone, betamethasone benzoate, chloroprednisone acetate, clocortolone acetate, descinolone acetonide, desoximetasone, dichlorisone acetate, difluprednate, flucloronide, flumethasone pivalate, flunisolide acetate, fluperolone acetate, fluprednisolone valerate, paramethasone acetate, prednisolamate, prednival, triamcinolone hexacetonide, cortivazol, formocortal and nivazol;

Pituitary hormones and their active derivatives or analogs such as corticotrophin, thyrotropin, follicle stimulating hormone, luteinising hormone and gonadotrophin releasing hormone;

Hypoglycemic agents such as insulin, chlorpropamide, glibenclamide, gliclazide, glipizide, tolazamide, tolbutamide and metformin;

Thyroid hormones such as calcitonin, thyroxine and liothyronine and antithyroid agents such as carbimazole and propylthiouracil;

Other miscellaneous hormone agents such as octreotide;

Pituitary inhibitors such as bromocriptine;

Ovulation inducers such as clomiphene;

Diuretics such as the thiazides, related diuretics and loop diuretics, bendroflumethiazide, chlorothiazide, chlorthalidone, dopamine, cyclopenthiazide, hydrochlorothiazide, indapamide, mefruside, methycholthiazide, metolazone, quinethazone, bumetamide, ethacrynic acid and furosemide and potassium sparing diuretics, spironolactone, amiloride and triamterene;

Antidiuretics such as desmopressin, lypressin and vasopressin including their active derivatives or analogs;

Obstetric drugs including agents acting on the uterus such as ergometrine, oxytocin and gemeprost;

Prostaglandins such as alprostadil, prostacyclin, dinoprost (prostaglandin F2-alpha) and misoprostol;

Antimicrobials including the cephalosporins such as cephalexin, cefoxytin and cephalothin;

Penicillins such as amoxycillin, amoxycillin with clavulanic acid, ampicillin, bacampicillin, benzathine penicillin, benzylpenicillin, carbenicillin, cloxacillin, methicillin, phenethicillin, phenoxymethylpenicillin, flucloxacillin, meziocillin, piperacillin, ticarcillin and azlocillin;

Tetracyclines such as minocycline, chlortetracycline, tetracycline, demeclocycline, doxycycline, methacycline and oxytetracycline and other tetracycline-type antibiotics;

Aminoglycosides such as amikacin, gentamicin, kanamycin, neomycin, netilmicin and tobramycin;

Antifungals such as amorolfine, isoconazole, clotrimazole, econazole, miconazole, nystatin, terbinafine, bifonazole, amphotericin, griseofulvin, ketoconazole, fluconazole and flucytosine, salicylic acid, fezatione, ticlatone, tolnaftate, triacetin, zinc, pyrithione and sodium pyrithione;

Quinolones such as nalidixic acid, cinoxacin, ciprofloxacin, enoxacin and norfloxacin;

Sulphonamides such as phthalysulphthiazole, sulfadoxine, sulphadiazine, sulphamethizole and sulphamethoxazole;

Sulphones such as dapsone;

Other miscellaneous antibiotics such as chloramphenicol, clindamycin, erythromycin, erythromycin ethyl carbonate, erythromycin estolate, erythromycin glucepate, erythromycin ethylsuccinate, erythromycin lactobionate, roxithromycin, lincomycin, natamycin, nitrofurantoin, spectinomycin, vancomycin, aztreonain, colistin IV, metronidazole, tinidazole, fusidic acid, trimethoprim, and 2-thiopyridine N-oxide; halogen compounds, particularly iodine and iodine compounds such as iodine-PVP complex and diiodohydroxyquin, hexachlorophene; chlorhexidine; chloroamine compounds; and benzoylperoxide;

Antituberculosis drugs such as ethambutol, isoniazid, pyrazinamide, rifampicin and clofazimine;

Antimalarials such as primaquine, pyrimethamine, chloroquine, hydroxychloroquine, quinine, mefloquine and halofantrine;

Antiviral agents such as acyclovir and acyclovir prodrugs, famcyclovir, zidovudine, didanosine, stavudine, lamivudine, zalcitabine, saquinavir, indinavir, ritonavir, n-docosanol, tromantadine and idoxuridine;

Anthelmintics such as mebendazole, thiabendazole, niclosamide, praziquantel, pyrantel embonate and diethylcarbamazine;

Cytotoxic agents such as plicainycin, cyclophosphamide, dacarbazine, fluorouracil and its prodrugs, methotrexate, procarbazine, 6-mercaptopurine and mucophenolic acid;

Anorectic and weight reducing agents including dexfenfluramine, fenfluramine, diethylpropion, mazindol and phentermine;

Agents used in hypercalcaemia such as calcitriol, dihydrotachysterol and their active derivatives or analogs;

Antitussives such as ethylmorphine, dextromethorphan and pholcodine;

Expectorants such as carbolcysteine, bromhexine, emetine, quanifesin, ipecacuanha and saponins;

Decongestants such as phenylephrine, phenylpropanolamine and pseudoephedrine;

Bronchospasm relaxants such as ephedrine, fenoterol, orciprenaline, rimiterol, salbutamol, sodium cromoglycate, cromoglycic acid and its prodrugs, terbutaline, ipratropium bromide, salmeterol and theophylline and theophylline derivatives;

Antihistamines such as meclozine, cyclizine, chlorcyclizine, hydroxyzine, brompheniramine, chlorpheniramine, clemastine, cyproheptadine, dexchlorpheniramine, diphenhydramine, diphenylamine, doxylamine, mebhydrolin, pheniramine, tripolidine, azatadine, diphenylpyraline, methdilazine, terfenadine, astemizole, loratidine, acrivastine, cinnarizine and cetirizine;

Local anaesthetics such as bupivacaine, amethocaine, lignocaine, lidocaine, cinchocaine, dibucaine, mepivacaine, prilocalne, etidocaine, veratridine (specific c-fiber blocker) and procaine;

Stratum corneum lipids, such as ceramides, cholesterol and free fatty acids, for improved skin barrier repair;

Neuromuscular blocking agents such as suxamethonium, alcuronium, pancuronium, atracurium, gallamine, tubocurarine and vecuronium;

Smoking cessation agents such as nicotine, bupropion and ibogaine;

Dermatological agents, such as vitamins A, C, B₁, B₂, B₆, B₁₂ₐ and E, vitamin E acetate and vitamin E sorbate, vitamin K;

Allergens for desensitisation such as house, dust or mite allergens;

Nutritional agents, such as vitamins, essential amino acids and fats;

Keratolytics such as the alpha-hydroxy acids, glycolic acid and salicylic acid;

Anti-protozoal agents, nitroimidazoles such as metronidazole;

Opiate antagonists and agonists such as naltrexone, naloxone, cyclazocine, metazocine, morphine, oxymorphone, methadone, fentanyl, sufentanil, alfentanil, buprenorphine, pentazocine and nalorphine;

Bone active agents including bisphosphonates such as alendronate, cimadronate, clodronate, etidronate, ibandronate, neridronate, olpadronate, pamidronate, risedronate, tiludronate, incadronate, [1-hydroxy-3-(1-pyrrolidinyl)-propylidene]bisphosphonate, [1-hydroxy-2-imidazo-(1,2-a) pyridin-3-ylethylidene]bis-phosphonate and zoledronate;

Compounds having antiprogestinic properties, e.g. such as those described in WO 01/47490;

Antihyperlipemic agents such as bezafibrate, fenofibrate, colestipol and statins.

Other pharmacologically active agents that may be used include anti-bacterial agents, anti-diabetics, anti-epileptics, anti-muscarinic agents, anti-neoplastic agents, erectile dysfunction improvement agents, immunosuppressants, anti-protozoal agents, β-blockers, anti-parkinsonian agents, gastro-intestinal agents, lipid regulating agents, cox-2-inhibitors, leukotriene inhibitors, macrolides, protease inhibitors, anti-osteoporosis agents anti-obesity agents, cognition enhancers, anti-urinary incontinence agents, anti-benign prostate hypertrophy agents, thrombin inhibitors, antithrombogenic agents, thrombolytic agents, fibrinolytic agents, vasospasm inhibitors, calcium channel blockers, inhibitors of surface glycoprotein receptors, antiplatelet agents, antimitotics, microtubule inhibitors, antisecretory agents, actin inhibitors, remodeling inhibitors, antisense nucleotides, antimetabolites, antiproliferatives, anticancer chemotherapeutic agents, growth hormone antagonists, growth factors, radiotherapeutic agents, peptides, proteins, enzymes, extracellular matrix components, free radical scavengers, chelators, antioxidants, antipolymerases, photodynamic therapy agents, gene therapy agents, drugs for vertigo, drugs for the central nervous system, drugs for the autonomic nervous system, autonomic ganglionic blockers, drugs for the peripheral nervous system, ophthalmic drugs, drugs for sense-organs, cardiacs, diuretics, vasoreinforcements, vasoconstrictors, antiarteriosclerotics, circulatory drugs, respiratory stimulants, drugs for respiratory organs, peptic ulcer drugs, stomachic digestants, antacids, cathartics, cholagogues, digestive drugs, urinary tract disinfectants, uterotonics, urogenital drugs, drugs for anus diseases, nutritive roborants, drugs for blood or body fluid, drugs for hepatic diseases, antidotes, habitual intoxication drugs, antipodagrics, enzyme preparations, cell activation drugs, antitumor agents, α-adrenergic blockers, cholinesterase inhibitors, anti-angiogenesis factors, anti-psoriatic agents, anti-diarrhoeals, anti-leukemic drugs, anti-aids drugs, drugs for dementia, angiotensin inhibitors, α- and β-agonists, wound-healing promoters, calcium antagonists, pancreatic hormones, spasmolytics, cardiovascular agents, inotropic agents, gonadotropins, symphatomimetic agents, antifungals, neurotrophic factors, proton pump inhibitors, antipruritics, anti-addiction drugs, histamin-receptor antagonists, immunosuppressants and immunostimulants.

In this specification, except where the context requires otherwise, the words “comprise”, “comprises” and “comprising” means “include”, “includes” and “including”, respectively. That is, when the invention is described or defined as comprising specified features, various embodiments of the same invention may also include additional features. Also the reference signs should not be construed as limiting the claims.

The invention is described below in greater detail by the following, non-limiting drawings and examples.

### BRIEF DESCRIPTION OF THE DRAWINGS

FIGS. 1a and 1b illustrate a first embodiment of the invention.

FIG. 2 illustrates a second embodiment of the invention.

FIG. 3 illustrates a third embodiment of the invention.

FIG. 4 illustrates a fourth embodiment of the invention.

FIG. 5 illustrates a fifth embodiment of the invention.

FIG. 6 illustrates a sixth embodiment of the invention.

FIG. 7 illustrates a seventh embodiment of the invention.

FIG. 8 illustrates an eighth embodiment of the invention.

FIG. 9 illustrates the results of Example 1.

FIG. 10 illustrates the results of Example 2.

FIG. 11 illustrates the results of Example 3.

FIGS. 12, 13 and 14 illustrate the results of Example 4.

### DETAILLED DESCRIPTION OF THE DRAWINGS

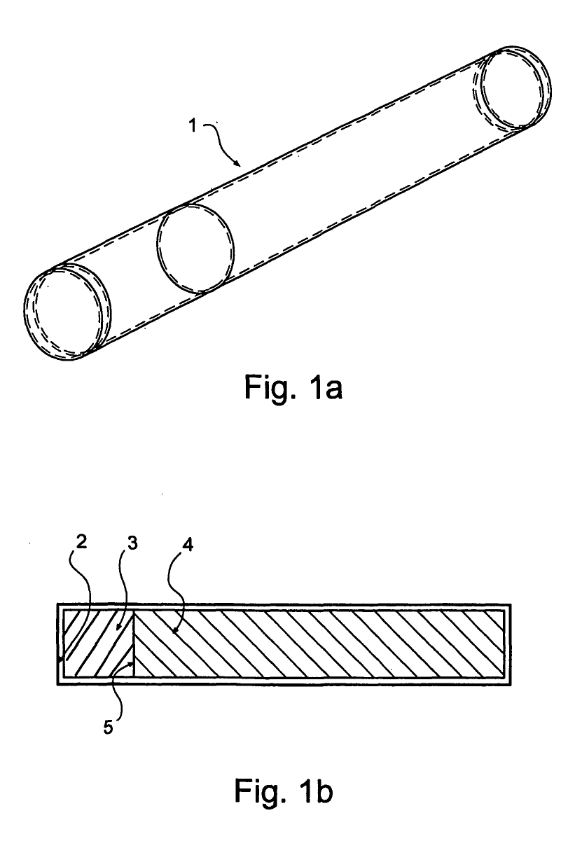

FIGS. 1a and 1b illustrate a first embodiment of the invention. In FIG. 1a, an implant 1 according to the invention is shown. FIG. 1b shows the same implant with further details. The implant is encased in a membrane 2 and its core consists of two parts 3 and 4 each comprising a different therapeutically active agent. A further separation membrane 5 separates the two parts 3 and 4.

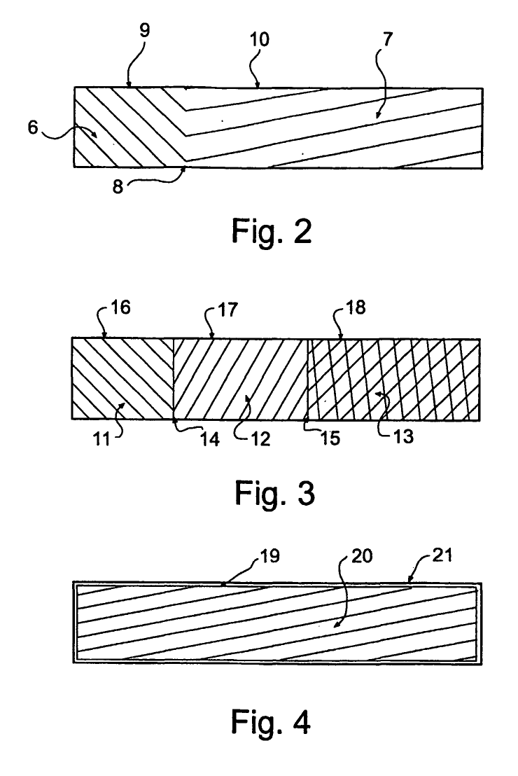

FIG. 2 illustrates a second embodiment of the invention. The system shown may be either an implant or part of an intrauterine, intracervical or intravaginal system. It consists of a core comprising two parts 6 and 7, each of them comprising a therapeutically active agent. In this embodiment, there is no membrane between the two parts (as can be seen at 8), but the elastomer compositions used in said two parts are different. The core is encased in a membrane consisting of two different elastomers, 9 and 10.

FIG. 3 illustrates a third embodiment of the invention. The system comprises three parts 11, 12 and 13, separated by separation membranes 14 and 15, the separation membrane 14 being permeable to the active agent contained in part 11 and impermeable to the active agent contained in part 12, and the separation membrane 15 being impermeable to the active agents contained in parts 12 and 13. Said part 13 contains two different active agents. The system is further encased in a membrane consisting of three parts 16, 17 and 18.

FIG. 4 illustrates a fourth embodiment of the invention. The system consists of a core 20 comprising three active agents, encased in a first membrane 19 and further in a second membrane 21, thicker that the first membrane 19.

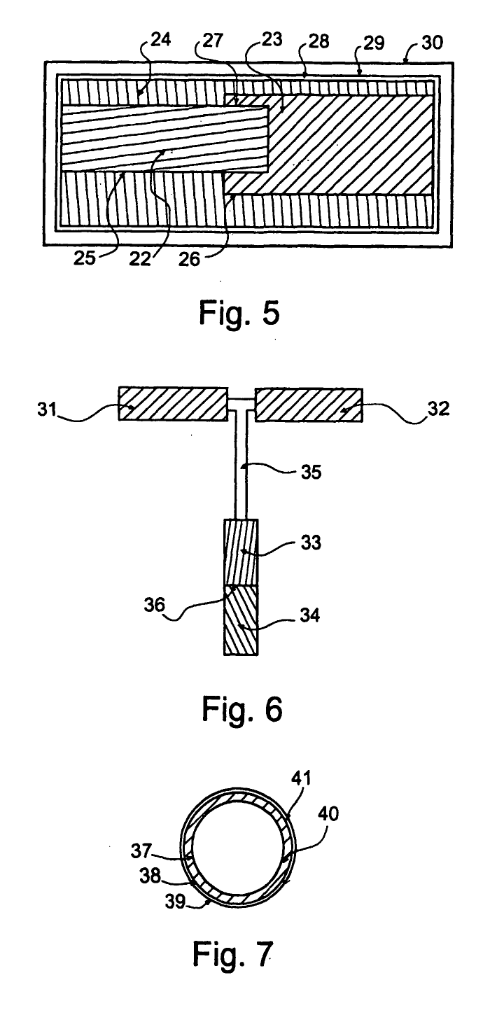

FIG. 5 illustrates a fifth embodiment of the invention. The core of the system consists of three parts 22, 23 and 24. The part 23 encases the part 22 partly and the part 24 encases both parts 22 and 23. The parts 22 and 23 are separated by a separation membrane 27, the parts 23 and 24 by a separation membrane 26 and parts 22 and 24 by a separation membrane 25. The core is then encased by a first membrane 28, a second membrane 29 and a third membrane 30, said third membrane being thicker than the first and second membranes. The distances between the membranes are exaggerated for clarity reasons.

FIG. 6 illustrates a sixth embodiment of the invention. The system is a T-shaped intra-uterine system comprising a body 35. The core consists of four parts 31, 32, 33 and 34. Each core is encased in a membrane. The parts 31 and 32 of the core are separated from each and from part 33 by a space. The parts 33 and 34 are adjacent and separated by a separation membrane 36.

FIG. 7 illustrates a seventh embodiment of the invention. The system is a intra-vaginal ring consisting of a first part of the core 40, encased in a second part of the core 41. The parts are separated by a separation membrane 38 and the inner surface of part 40 and the outer surface of part 41 are encased in membranes 37 and 39, respectively.

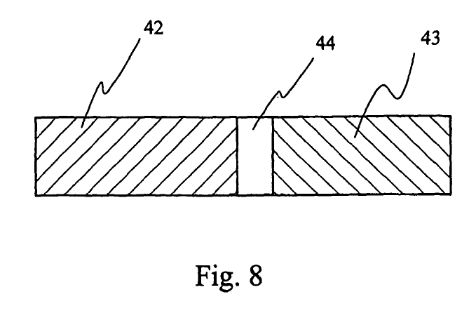

FIG. 8 illustrates an eighth embodiment of the invention. The core of the system comprises two parts 42 and 43 separated by a space 44.

### EXPERIMENTAL PART

The invention is further illustrated by the following, non-limiting examples.

### Example 1

An implant comprising levonorgestrel at a target release rate of 50 μg/day and estradiol at a target release rate of 10 μg/day was prepared.

The implant structure was as disclosed in FIG. 2. The first part of the core consisted of PDMS comprising levonorgestrel and the length was 35 mm. The second part of the core consisted of PEO-PDMS having 50% of PEO, comprising estradiol, and the length was 8 mm.

The core parts were encased in a membrane consisting of PEO-PDMS in a ratio of 10:90. The thickness of the membrane was 0.2 mm and the outer diameter of the implant 2.48 mm.

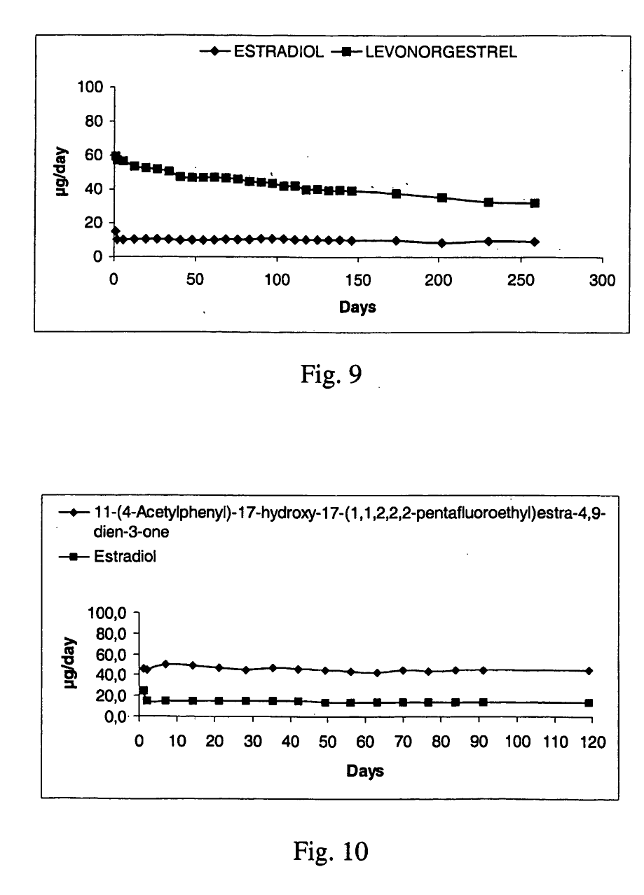

The release rates obtained are illustrated in FIG. 9, wherein the squares illustrate the release rate of estradiol and the lozenges represent the release rate of levonorgestrel. It can be seen that the target release rate of estradiol was obtained and that the release rate of levonorgestrel was from 60 to 40 μg/day instead of the 50 μg/day targeted.

### Example 2

An implant according to the Example 1 was prepared, using as active agents 11-(4-Acetylphenyl)-17-hydroxy-17-(1,1,2,2,2-pentafluoroethyl)estra-4,9-dien-3-one (an antiprogestin) at a target release rate of 50 μg/day and estradiol at a target release rate of 10 μg/day.

The implant structure was as disclosed in FIG. 2. The first part of the core consisted of PEO-PDMS in a ratio of 50:50 comprising compound 1 and the length was 34 mm. The second part of the core consisted of PEO-PDMS having 50% of PEO, comprising estradiol, and the length was 6 mm.

The core parts were encased in a membrane consisting of PEO-PDMS in a ratio of 20:80. The thickness of the membrane was 0.2 mm and the outer diameter of the implant 2.48 mm.

The release rates obtained are illustrated in FIG. 10, wherein the lozenges illustrate the release rate of estradiol and the squares represent the release rate of compound 1. It can be seen that the target release rates were obtained.

### Example 3

An implant according to the Example 1 was prepared, using as active agents gestodene and estradiol.

The implant structure was as disclosed in FIG. 2. The first part of the core consisted of PDMS comprising gestodene and the length was 13 mm. The second part of the core consisted of PEO-PDMS having 50% of PEO, comprising estradiol, and the length was 30 mm.

The core parts were encased in a membrane consisting of PDMS and methyltrifluoropropyl-methylvinyl siloxane in a ratio of 70:30. The thickness of the membrane was 0.23 mm and the outer diameter of the implant 2.48 mm.

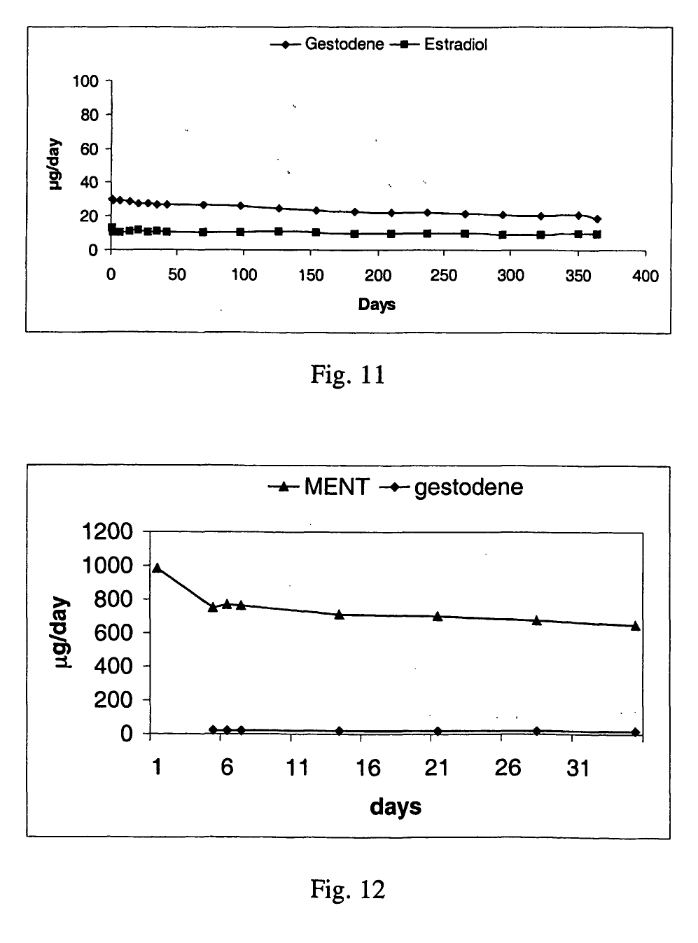

The release rates obtained are illustrated in FIG. 11, wherein the lozenges illustrate the release rate of gestodene and the squares represent the release rate of estradiol.

### Example 4

Implants according to the Example 1 were prepared using as active agents 7-α-methyl-19-nortestosterone (MENT) and gestodene.

The implant structure was as disclosed in FIG. 2. The first part of the core consisted of Pt-catalysed PDMS comprising 60 weight-% MENT having a length of 44 mm and a diameter of 3.0 mm. The second part of the core consisted of peroxide-catalysed PDMS comprising 50 weight-% gestodene having a length of 12 mm and a diameter of 3.0 mm.

Both core parts were encapsulated by a membrane consisting of a mixture of PDMS and trifluoropropyl modified PDMS. The fluoro-content in the membrane varied from 55 weight-% to 75 weight-%. The thickness of the membrane was 0.25 or 0.35 mm and thus the outer diameter of the implant 3.5 or 3.7 mm, respectively.

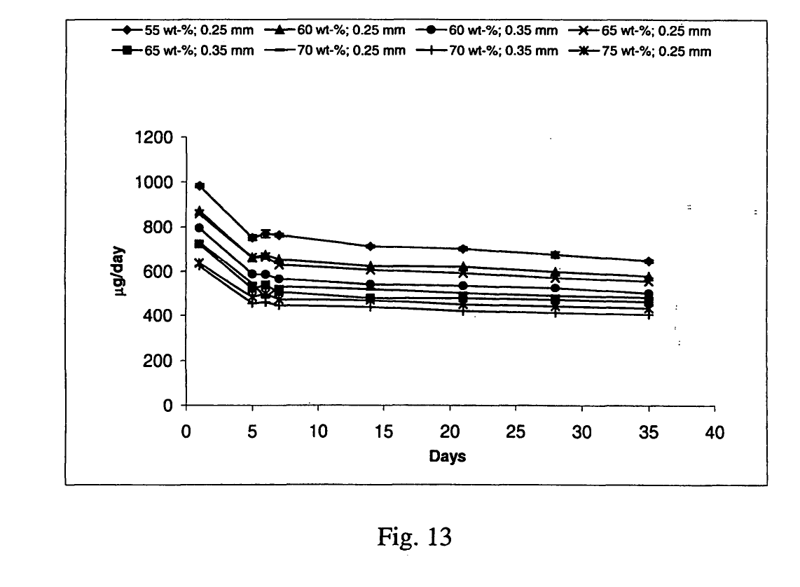

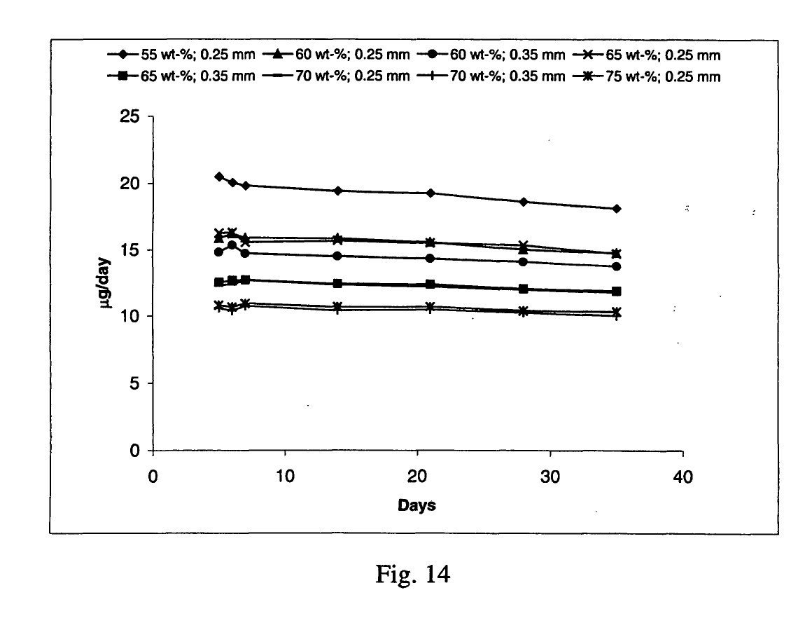

The release rates obtained are illustrated in FIGS. 12 to 14, wherein FIG. 12 illustrates the release rate of MENT and gestodene from an implant wherein the fluoro-content of the membrane was 55 wt-% and the thickness of said membrane was 0.25 mm. MENT is represented by the trinagles and gestodene by the lozenges. It can be seen that the release rates of both active agents are essentially constant over time. FIGS. 13 and 14 illustrate the release rates of MENT and gestodene, respectively, in function of the fluoro-content (55-75 wt-%) and the thickness (0.25 or 0.35 mm) of the membrane. It can be seen that the release rate can be quite accurately adjusted by the proper choice of the fluoro-content and the thickness. For example, in FIG. 14, one can see that the release rate of gestodene from an implant encased in a membrane having a fluoro-content of 60 wt-% and a thickness of 0.25 mm (represented by the triangles) is higher than that from an implant encased in a membrane having a fluoro-content of 60 wt-% and a thickness of 0.35 mm (represented by the spheres).

1-27. (Canceled)

28. A delivery system comprising a core consisting of one part, and a membrane encasing said core, wherein said core and membrane consist essentially of a same or different elastomer composition, and wherein said core comprises at least two therapeutically active agents each having a release rate.

29. A delivery system comprising a core, and a membrane encasing said core, wherein said core and membrane consist essentially of a same or different elastomer composition, and wherein said core consists of at least a first and a second part, each part comprising at least one therapeutically active agent each having a release rate, and wherein it further comprises at least one separation membrane separating at least two of the said at least two parts of the core, said separation membrane consisting essentially of an elastomer composition.

30. The delivery system of claim 29, wherein said first and second (and further) parts are adjacent to one another.

31. The delivery system of claim 29, wherein said second (or further) part encases at least partly said first (or second or further) part.

32. The delivery system of claim 28, wherein the membrane comprises at least two layers.

33. The delivery system of claim 29, wherein the membrane comprises at least two layers.

34. The delivery system of claim 32, wherein the thickness of the layers are different.

35. The delivery system of claim 33, wherein the thickness of the layers are different.

36. The delivery system of claim 29, further comprising a space separating at least two of the said at least two parts of the core.

37. The delivery system of claim 29, wherein said at least one separation membrane is permeable to at least one of the therapeutically active agents.

38. The delivery system of claim 29, wherein said at least one separation membrane is impermeable to the therapeutically active agents.

39. The delivery system of claim 28, wherein said elastomer compositions are the same or different, and said elastomer compositions are selected from the group consisting of
an elastomer composition comprising poly(dimethylsiloxane),
an elastomer composition comprising a siloxane-based elastomer comprising 3,3,3-trifluoropropyl groups attached to the Si atoms of the siloxane units,
an elastomer composition comprising poly(alkylene oxide) groups, said poly(alkylene oxide) groups being present as alkoxy-terminated grafts or blocks linked to the polysiloxane units by silicon-carbon bonds, or as a mixture of these forms; and
a combination of at least two thereof.

40. The delivery system of claim 29, wherein said elastomer compositions are the same or different, and said elastomer compositions are selected from the group consisting of
an elastomer composition comprising poly(dimethylsiloxane),
an elastomer composition comprising a siloxane-based elastomer comprising 3,3,3-trifluoropropyl groups attached to the Si atoms of the siloxane units,
an elastomer composition comprising poly(alkylene oxide) groups, said poly(alkylene oxide) groups being present as alkoxy-terminated grafts or blocks linked to the polysiloxane units by silicon-carbon bonds, or as a mixture of these forms; and
a combination of at least two thereof.

41. The delivery system of claim 39, wherein, in the siloxane-based elastomer, 1 to approximately 50% of the substituents attached to the Si-atoms of the siloxane units are 3,3,3-trifluoropropyl groups.

42. The delivery system of claim 40, wherein, in the siloxane-based elastomer, 1 to approximately 50% of the substituents attached to the Si-atoms of the siloxane units are 3,3,3-trifluoropropyl groups.

43. The delivery system of claim 39, wherein the poly(alkylene oxide) groups are poly(ethylene oxide) groups.

44. The delivery system of claim 40, wherein the poly(alkylene oxide) groups are poly(ethylene oxide) groups.

45. The delivery system of claim 29, wherein the elastomer compositions of the said at least two parts of the core are identical.

46. The delivery system of claim 29, wherein the elastomer compositions of the said at least two parts of the core are different.

47. The delivery system of claim 29, wherein the elastomer compositions of the membranes of the said at least two parts are identical.

48. The delivery system of claim 29, wherein the elastomer compositions of the membranes of the said at least two parts are different.

49. The delivery system of claim 33, wherein the elastomer compositions of the different layers of the membrane are identical.

50. The delivery system of claim 33, wherein the elastomer compositions of the different layers of the membrane are different.

51. The delivery system of claim 34, wherein the elastomer compositions of the different layers of the membrane are identical.

52. The delivery system of claim 34, wherein the elastomer compositions of the different layers of the membrane are different.

53. The delivery system of claim 29, wherein the elastomer compositions of the separation membranes are identical.

54. The delivery system of claim 29, wherein the elastomer compositions of the separation membranes are different.

55. The delivery system of claim 28, wherein the release rates of the at least two therapeutically active agents are identical.

56. The delivery system of claim 29, wherein the release rates of the at least two therapeutically active agents are identical.

57. The delivery system of claim 28, wherein the release rates of the at least two therapeutically active agents are different.

58. The delivery system of claim 29, wherein the release rates of the at least two therapeutically active agents are different.

59. The delivery system of claim 28, wherein the release rates are determined by the core and the membrane.

60. The delivery system of claim 29, wherein the release rates are determined by the core and the membrane.

61. The delivery system of claim 28, wherein the release rates are determined by the membrane.

62. The delivery system of claim 29, wherein the release rates are determined by the membrane.

63. The delivery system of claim 28, wherein said system is an implant.

64. The delivery system of claim 29, wherein said system is an implant.

65. The delivery system of claim 28, wherein said system is selected from the group consisting of an intrauterine system, an intracervical system and an intravaginal system.

66. The delivery system of claim 29, wherein said system is selected from the group consisting of an intrauterine system, an intracervical system and an intravaginal system.

67. The delivery system of claim 28, wherein said therapeutically active agent is a hormone.

68. The delivery system of claim 29, wherein said therapeutically active agent is a hormone.
